(19) United States
(12) Patent Application Publication
(10) Pub. No.: US 2019/0298243 A1
(43) Pub. Date: Oct. 3, 2019

(54) SYSTEM, METHOD AND COMPUTER PROGRAM FOR QUANTIFYING PHYSICAL FATIGUE OF A SUBJECT

(71) Applicant: KONINKLIJKE PHILIPS N.V., EINDHOVEN (NL)

(72) Inventors: Alberto Giovanni BONOMI, Eindhoven (NL); Francesco SARTOR, Eindhoven (NL); Gabriele PAPINI, Eindhoven (NL)

(21) Appl. No.: 16/347,405

(22) PCT Filed: Nov. 3, 2017
(86) PCT No.: PCT/EP2017/078248

§ 371 Date: May 3, 2019

(30) Foreign Application Priority Data
Nov. 7, 2016 (EP) 16197478.7

(51) Int. Cl. A61B 5/16; A61B 5/11; A61B 5/024; G06F 3/01

(52) U.S. Cl. CPC A61B 5/165; A61B 5/1118; G06F 2203/011; G06F 3/015; A61B 5/02405

(57) ABSTRACT

A system, method and corresponding computer program for quantifying physical fatigue are provided, the system comprising: a physiological measure providing unit (20) for providing a physiological measure of the subject, a fatigue index determination unit (200) for determining a fatigue index of the subject. The fatigue index determination unit (200) comprises a first fatigue index determination subunit (210) for determining a first fatigue index based on the physiological measure and a second fatigue index determination subunit (240) for determining a second fatigue index based on the physiological measure. The first fatigue index and the second fatigue index have a respectively different characteristic based on the physiological measure. The system, method and corresponding computer program improve the quantifying of physical fatigue of a subject.

### FIELD OF THE INVENTION

The present invention relates to the field of quantifying physical fatigue of a subject. In particular, it relates to a system, a method and a computer program for quantifying physical fatigue of a subject. It finds particular application in improving sports performance, in particular in the field of recovery after exercising. However, it is to be understood that the present invention also finds applications in other fields and is not necessarily limited to the above mentioned application.

### BACKGROUND OF THE INVENTION

Physical fatigue can be described as a physiological phenomenon occurring during and after exercise, which results in deterioration of physical performance of a subject.

From U.S. Pat. No. 7,192,401 B2 a method for determining accumulated body fatigue index (BFI) is known, wherein one or more parameters from the measurement of one or more signals are obtained sequentially as input and these parameters are information on the intensity of physical activity. The BFI has a predetermined initial value and the next BFI value is always a sum of the BFI value and a difference. The difference is a combination of upslope and optional downslope components of BFI determined with the said parameters, and the upslope component and the optional downslope component are each determined with a function, which is scaled by a preset physiological character.

This method has several drawbacks, which result in unsatisfactory quantification and estimation of physical fatigue in various situations. For instance, the known method will underestimate physical fatigue for long term, low intensity activities, such as long walks. Further, recovery from physical fatigue, i.e. a decrease of a corresponding index, may be different depending on the origin of the corresponding fatigue, e.g. a high intensity exercise, e.g. resulting in a high concentration of lactate, may show a different recovery profile as compared to the fatigue mainly generated by dehydration and hyperthermia, i.e. fatigue resulting from low intensity exercise.

US 2015/0250417 A1 discloses a stamina monitoring device, comprising: a sensor module to sense a physiological signal; an user interface to receive a biological information; a storage to store the biological information and at least one mathematical model; a processing module to execute the at least one mathematical model to do the following: estimating an anaerobic energy and an aerobic energy based on the physiological signal and the biological information; combining the anaerobic energy and the aerobic energy into a stamina level.

US 2014/0018945 A1 concerns determining physiological training effect of a physiological performance of a person by monitoring the performance using one or more performance monitoring means in order to obtain performance data, and, according to one aspect of the invention, determining, using computing means capable of utilizing the performance data, a third training effect parameter describing a third physiological effect of the performance using a third determination method, the third physiological effect being a combination effect of the first and second physiological effects which are different from each other and are descriptive of different physiological effects of training, such as homeostatic disturbance and cumulative physiological load, respectively.

The article “Modeling Human Performance”, retrieved from the Internet, May 16, 2016, URL: http://fellrnr.com/wiki/Modeling Human Performance, describes a number of approaches to modeling how training changes performance and these models have some obvious value in optimizing a training routine, especially for tapering. The models assume that given training stress, also known as “training impulse” or TRIMP (TRaining IMPulse), has both a positive and a negative effect. The positive effect is called “fitness”, and the negative effect is called “fatigue”, and they are combined to provide a value of “performance”.

The article “TRIMP—Fellrnr.com, Running tips”, retrieved from the Internet, Mar. 19, 2005, URL: http://fellrnr.com/wiki/TRIMP, describes various ways of measuring the impact of exercise and these measures are often termed “Training Impulse”, or TRIMP (TRaining IMPulse). TRIMP can be used to model human performance to evaluate the effect of training over time.

### SUMMARY OF THE INVENTION

It is thus an object of the present invention to provide an improved system, method and corresponding computer program for quantifying physical fatigue of a subject.

In a first aspect, a system for quantifying physical fatigue of a subject is provided. The system comprises a physiological measure providing unit for providing a physiological measure of the subject, a fatigue index determination unit for determining a fatigue index of the subject, wherein the fatigue index determination unit comprises a first fatigue index determination subunit for determining a first fatigue index based on the physiological measure and a second fatigue index determination subunit for determining a second fatigue index based on the physiological measure. The first fatigue index and the second fatigue index have a respectively different characteristic based on the physiological measure.

Two independent fatigue indexes, namely the first fatigue index and the second fatigue index, which can be utilized for describing the metabolic fatigue accumulated during different intensities of activity, like jogging or sprints, and which can optimize different types of recovery, are employed.

Since the system according to this aspect is configured to determine a first fatigue index and a second fatigue index of the subject preferably for the same point in time, fatigue from different causes based on the physiological measure can be distinguished. For instance, fatigue from high intensity exercise, i.e. anaerobic exercise, can be distinguished from fatigue from aerobic exercise and different factors which influence physical fatigue in different exercise regions can be distinguished. Accordingly, for each contributing factor, such as dehydration, lactate accumulation, heat accumulation, vasodilatation, muscle damage and so on, a particular recovery profile can be determined. Thus, the system according to this aspect can lead to an improved managing of training programs for athletes by helping to avoid overtraining, to dose training intensity and, particularly, to estimate recovery time.

While the fatigue index can be determined as a combination of first fatigue index and second fatigue index, for instance a sum of both indexes, each of the first fatigue index and the second fatigue index can advantageously be considered alone, for instance, to determine an optimized recovery profile.

In an embodiment the physiological measure is indicative of an intensity of a physical activity of the subject. Such physiological measures include, without being limited, a heart rate of the subject, a respiration rate, respiration intensity, CO₂ concentration, a body temperature and blood markers, such as a lactate concentration or creatinine concentration, for instance. Preferentially, the physiological measure allows for a classification of the activity to an activity class.

In an embodiment the first fatigue index is an aerobic fatigue index indicative of an aerobic fatigue of the subject and the second fatigue index is an anaerobic fatigue index indicative of an anaerobic fatigue of the subject. The aerobic fatigue index and the anaerobic fatigue index can be utilized for describing the metabolic fatigue accumulated during different intensities of activity, like jogging or sprints, and to optimize the respective fatigue index for different types of recovery. However, also other examples of a first and second fatigue index which have different characteristics based on the physiological measure can be used in this aspect of the present invention.

The physiological measure providing unit can comprise a storing unit, in which the physiological measure of the subject is stored already. However, the physiological measure providing unit can also be a receiving unit for receiving a physiological measure from a measurement unit and for providing the received physiological measurement signal. Moreover, the physiological measure providing unit can also be the measurement unit itself, wherein the physiological measure providing unit provides the measured physiological measure.

In an embodiment the first fatigue index determination subunit comprises a first fatigue index increment subunit for increasing the first fatigue index and a first fatigue index decrement subunit for decreasing the first fatigue index. The second fatigue index determination subunit comprises a second fatigue index increment subunit for increasing the second fatigue index and a second fatigue index decrement subunit for decreasing the second fatigue index. The first fatigue index decrement subunit and the second fatigue index decrement subunit are configured to determine the first fatigue index decrement and the second fatigue index decrement with a respectively different dependency on the physiological measure.

Just to give an example, while lactate accumulation can be predominant in high intensity, i.e. anaerobic, exercise, dehydration and hyperthermia can be the main contributors to aerobic fatigue. However, aerobic fatigue and anaerobic fatigue are just two examples of different types of fatigue and further types are contemplated.

Since the first fatigue index and the second fatigue index are determined by independent increment and decrement subunits, different factors contributing to different aspects of fatigue, such as aerobic fatigue and anaerobic fatigue, can be specifically addressed. Since the first and second fatigue indices have a different characteristic based on the physiological measure and since different predominant origins of fatigue can be linked with the physiological measure, a different recovery profile for the corresponding fatigue index can be applicable. More precisely and to give an example, in order to recover from a high lactate concentration, exercise at a low intensity can be favorable, while recovery time from a low intensity exercise, will be hindered by ongoing exercise, even at low intensity.

In an embodiment the system further comprises a weighting function determination unit for determining a weighting function based on the physiological measure. The weighting function is configured to determine a weight which increases with an activity intensity indicated by the physiological measure. The second fatigue index determination subunit is configured to determine the second fatigue index based on the physiological measure and the weighting function.

Preferentially, the first fatigue index increment can be the same as the second fatigue index increment but for the weight, i.e. the weighting function determining a weight which increases with the physiological measure. However, in other embodiments, the increment implementation can also substantially differ between first fatigue index increment subunit and second fatigue index increment subunit depending on the specific implementation.

Since the weighting function determines a weight, which increases with the activity intensity indicated by the physiological measure, and since the second fatigue index is determined based on the weighting function, higher intensity activity, e.g. anaerobic activity, contributes with a higher factor due to the higher weight. Preferentially, only activities resulting in a value of the physiological measure above a threshold contribute significantly to the second fatigue index.

The weighting function preferably comprises a sigmoid function, which shows a flex point that can be adapted to correspond approximately to a threshold, which allows a classification of the physiological measure. In one example, the threshold can be an anaerobic threshold, which can be set to, for instance, 80% of a maximal heart rate. In other embodiments, also other weighting functions, which increase with the physiological measure, are contemplated. For instance, such other weighting functions could comprise step functions and the like. Additionally or alternatively to the physiological measurement, this embodiment can further comprise at least one of the fitness parameter providing unit and the training impulse variable determination unit as described above and the weighting function can alternatively or additionally depend on the fitness parameter and/or the training impulse variable.

In an embodiment the system further comprises an exercise timer providing unit for providing an exercise time of the subject. The physiological measure providing unit is configured to provide the physiological measure corresponding to the provided exercise time.

The exercise timer providing unit is preferentially a unit for providing the exercise time of the subject, which corresponds to a time the subject is carrying out an activity. The activity can for instance be the subject walking, running, or engaging in any other physical activity, without being limited.

The exercise timer providing unit can comprise a timer, which provides the rest of the system, preferably the physiological measure providing unit, with a timer signal indicative of the exercise time of the subject.

Preferentially, the physiological measure providing unit and the exercise timer providing unit provide a set of a physiological measure and corresponding exercise time of the subject substantially in real time, i.e. during the exercising of the subject. In another example, a physiological measure of the subject and the corresponding exercise time is recorded during the exercising of the subject and the physiological measure providing unit and the exercise timer providing unit are configured to provide the previously recorded physiological measure and corresponding exercise time. Preferentially, the physiological measure, e.g. the heart rate, and corresponding exercise time can be stored in a database, a table, a list and so on in this embodiment.

At least one of the first and second fatigue index is preferentially determined for each provided exercise time based on a previous fatigue index, corresponding to a preceding physiological measure and corresponding exercise time. The difference in time between a preceding physiological measure and the provided physiological measure can be described as a sampling frequency for the fatigue index. In an embodiment this sampling frequency is predefined and constant, however, in other embodiments the sampling frequency can also be set dynamically and/or depend on the physiological measure.

In an embodiment the system further comprises a delay time determination unit for determining a delay time based on the physiological measure. At least one of the first and second fatigue index increment subunits is configured to increase the fatigue index in case the exercise time exceeds the delay time only.

Since at least one of the fatigue index increment subunits is configured to increase the respective fatigue index in case the exercise time exceeds the delay time only, and since the fatigue index is increased based on the physiological measure, a different beginning of the exercise and the onset of fatiguing of the subject can be taken into account. The invention is thereby based on the finding that accumulation of fatigue does not start with the beginning of an exercise. Preferably, the delay time is determined such as to correspond to the time required to perceive and undergo fatigue. Preferentially, the delay time determination unit thereby determines the delay time based on the physiological measure, which correlates with the intensity of the activity of the subject. The system according to the invention can therefore also account for a physiological fatigue of long lasting and low intensity activities of the subject.

In an embodiment the system further comprises a fitness parameter providing unit for providing a fitness parameter representative of a fitness of the subject.

The fitness parameter providing unit can in one embodiment provide a previously determined fitness parameter, for instance, a fitness parameter, which is stored on the system itself or in a remote entity, for example a database storing also further parameters of the subject, such as preferably a maximum heart rate and a resting heart rate or other parameters related to physiological measures. However, in other embodiments, the fitness parameter providing unit can also comprise a fitness parameter determination unit for determining the fitness parameter, for instance through measurements carried out on the subject. In an embodiment the fitness parameter comprises maximal oxygen consumption (VO2ₘₐₓ) of the subject. The maximal oxygen consumption can be either an absolute rate in, for example, liters of oxygen per minute or a relative rate in, for example, milliliters of oxygen per kilogram of body mass per minute. Particularly, the relative rate can be advantageously employed as a fitness parameter, which is representative of a fitness of the subject, since it can readily be compared among different subjects without the influence of different subjects' body masses. However, also other fitness parameters as known in the art are contemplated. For example, a result of a questionnaire, in which the subject answers fitness related questions, can be taken as a fitness parameter.

Preferably, the delay time determination unit is configured to determine the delay time based on the fitness parameter. Since the delay time is determined based on the fitness parameter, which itself is representative of a fitness of the subject, the delay time accounts for the fitness of the subject. More specifically, since the fitness of the subject is taken into consideration for the delay time, a delay time can for instance be determined to be shorter for subjects with a lower fitness, since it can be assumed that physical fatigue starts to accumulate earlier with subjects with lower levels of fitness, than it does for subjects with higher levels of fitness. Expressed differently, subjects with an increased level of fitness can experience the effect of physical fatigue later after starting of an exercise, than subjects with a lower level of fitness will.

Additionally or alternatively, at least one of the first fatigue index decrement subunit and the second fatigue index decrement subunit is preferably configured to decrease the fatigue index based on the fitness parameter. It can be assumed that a subject having a higher fitness, based on the fitness parameter, will experience a more efficient removal of physical fatigue. Accordingly, since a fatigue index decrement subunit decreases the fatigue index based on the fitness parameter, a more accurate fatigue index determination can be achieved.

In an embodiment the physiological measure providing unit comprises a heart rate providing unit for providing a heart rate of the subject, and the system further comprises a resting heart rate providing unit for providing a resting heart rate of the subject, a maximum heart rate providing unit for providing a maximum heart rate of the subject, and a heart rate reserve determination unit for determining a heart rate reserve based on the resting heart rate and the maximum heart rate. The heart rate providing unit is further configured to provide the heart rate of the subject in proportion to the heart rate reserve as a heart rate reserve percentage.

The heart rate providing unit can comprise a storing unit, in which the heart rate of the subject is stored already in association with the corresponding exercise time. In this embodiment, the exercise timer providing unit can provide the exercise time stored in association with the heart rate. However, the heart rate providing unit can also be a receiving unit for receiving a heart rate signal from a heart rate measurement unit and for providing the received heart rate signal. Moreover, the heart rate providing unit can also be the heart rate measurement unit itself, wherein the heart rate providing unit provides the measured heart rate signal.

The resting heart rate and the maximum heart rate can be stored already in association with the subject and the system, for instance on a storage unit provided in the system itself or in connection with a database, wherein the resting heart rate providing unit and the maximum heart rate providing unit are configured to receive the resting heart rate and the maximum heart rate from the database. However, in other embodiments, the resting heart rate and the maximum heart rate can also be provided in a different way, such as estimated and provided based on the exercising of the subject.

Preferentially, the heart rate reserve is determined as a difference between maximum heart rate and resting heart rate. The heart rate reserve percentage is then a proportional position of the heart rate of the subject at the provided exercise time, i.e. at a particular current or previous point in time. A heart rate reserve percentage of 100% therefore preferentially corresponds to the subject exercising at maximum heart rate, wherein a heart rate reserve percentage of 0% preferentially corresponds to the subject being at rest, i.e. the heart rate corresponding to the resting heart rate.

Preferably, at least one of the first and second fatigue index increment subunits is configured to increase the respective first or second fatigue index based on the heart rate reserve percentage or parameters derived from the heart rate reserve percentage. Since the fatigue index increment subunit increases the fatigue index based on the heart rate reserve percentage, the subject's individual heart characteristics are employed in determining the fatigue index and the fatigue index can be determined more accurately.

Preferably, at least one of the first and second fatigue index decrement subunit is configured to decrease the respective first or second fatigue index based on at least one of the heart rate and the heart rate reserve percentage. Since the fatigue index decrement subunit decreases the fatigue index based on an exercise related parameter indicative of an intensity of the activity, e.g. on the heart rate or the heart rate reserve percentage, removal of physical fatigue can accurately be estimated.

In an embodiment the system further comprises a training impulse variable determination unit for determining a training impulse variable based on the physiological measure, in particular based on at least one of the heart rate and the heart rate reserve percentage. At least one of the first fatigue index increment subunit, the first fatigue index decrement subunit, the second fatigue index increment subunit and the second fatigue index decrement subunit is configured to respectively increment or decrement the fatigue index based on the training impulse variable.

Preferentially, the training impulse variable is a function of the actual intensity of the exercise, for instance represented by the physiological measure such as the heart rate, and on subject characteristics. One example of the training impulse variable has been described previously in Morton, R. H., Fitz-Clarke, J. R., & Banister, E. W. (1990). Modeling human performance in running J Appl Physiol (1985), 69(3), 1171-1177.

Preferentially, the fatigue index increment subunit directly sums the training impulse variable to the previous fatigue index or multiplies the training impulse variable with a value dependent on the sampling frequency, i.e. e.g. a time difference between a preceding physiological measure and a current physiological measure. Preferentially, the physiological measure is provided at a constant frequency, such as, for instance, each second. However, in other embodiments, the sampling frequency can also be different or dynamic, for instance, depending on the physiological measure itself.

In an embodiment the first fatigue index decrement subunit is configured to determine the first fatigue index decrement exponentially proportional to the inverse of the training impulse variable, and the second fatigue index decrement subunit is configured to determine the second fatigue index decrement exponentially proportional to the training impulse variable.

Since the first fatigue index decrement is determined exponentially proportional to the inverse of the training impulse variable and since the second fatigue index decrement is determined exponentially proportional to the training impulse variable, the first fatigue index decrement and the second fatigue index decrement have different dependencies on the training impulse variable. More precisely, while an increase in the training impulse variable would lead to a lower decrease in the first fatigue index, it would result in a higher decrease of the second fatigue index. Accordingly, the second fatigue index, which, for instance corresponds to a predominant lactate concentration, will be decreased in case the subject keeps on exercising with a moderate intensity. This in line with the scientific finding that motion aids in releasing lactate from the muscles. To the contrary, since the first fatigue index is indicative of a predominant dehydration and hyperthermia contribution, for instance, ongoing activity, even moderate intensity activities, does not support the recovery. Instead, in order to recover from a high first fatigue index, for instance from aerobic fatigue, the subject would be most efficiently be at rest.

In an embodiment the training impulse variable determination unit is configured to determine the training impulse variable based on a product of the heart rate reserve percentage and an exponential of the heart rate reserve percentage. The training impulse variable with this dependency has shown to reproduce the physiological effect of fatigue. However, in other embodiments, also different and deviating functional dependencies can be employed for the training impulse variable.

In an embodiment the training impulse variable determination unit is configured to determine the training impulse variable further based on at least one constant depending on a gender of the subject.

Preferentially, the constant can be a linear factor, i.e. multiplied linearly with the remainder of the training impulse variable. Alternatively or additionally, the constant can be an exponential constant, i.e. being a constant factor in the exponent to be multiplied with the heart rate reserve percentage, of which the exponential is determined.

Advantageously, since the constant depending on the gender of the subject and the heart rate reserve percentage, which considers subject specific heart related parameters, is employed, an individualized variable indicative of the training impulse of the subject can be determined.

In a further aspect, a system for quantifying physical fatigue of a subject is provided. The system comprises an exercise timer providing unit for providing an exercise time of the subject, a physiological measure providing unit for providing a physiological measure of the subject corresponding to the provided exercise time, a fatigue index determination unit for determining a fatigue index of the subject, a delay time determination unit for determining a delay time based on the physiological measure. The fatigue index determination unit comprises a fatigue index increment subunit for increasing the fatigue index based on the physiological measure. The fatigue index increment subunit is configured to increase the fatigue index in case the exercise time exceeds the delay time only.

The system according to this aspect can be combined with any of the embodiments described above. Although the first and second fatigue indices are not mandatory in this aspect and a single fatigue index can be sufficient, a combination with one, more or all of the above described units can lead to a combined system which improves the quantification of physical fatigue of the subject.

Since the fatigue index increment subunit is configured to increase the fatigue index in case the exercise time exceeds the delay time only, and since the fatigue index is increased based on the physiological measure, a different beginning of the exercise and the onset of fatiguing of the subject can be taken into account. The invention is thereby based on the finding that accumulation of fatigue does not start with the beginning of an exercise. Preferably, the delay time is determined such as to correspond to the time required to perceive and undergo fatigue. Preferentially, the delay time determination unit thereby determines the delay time based on the physiological measure, which correlates with the intensity of the activity of the subject. The system according to the invention can therefore also account for a physiological fatigue of long lasting and low intensity activities of the subject.

In an embodiment the fatigue index determination unit further comprises a fatigue index decrement subunit for decreasing the fatigue index based on at least one of the heart rate and the heart rate reserve percentage. Since the fatigue index decrement subunit decreases the fatigue index based on an exercise related parameter indicative of an intensity of the activity, e.g. on the heart rate or the heart rate reserve percentage, removal of physical fatigue can accurately be estimated.

In an embodiment the system further comprises a training impulse variable determination unit for determining a training impulse variable based on at least one of the heart rate and the heart rate reserve percentage. The fatigue index decrement subunit is configured to decrement the fatigue index based on the training impulse variable.

Since the fatigue index is decremented based on the training impulse variable, a type of exercise and the relative intensity and impact on subject physiology can be accounted for the decrement of the fatigue index. Preferentially, the training impulse variable determination unit can be combined with one, more or all the specific embodiments of the training impulse variable determination unit discussed above.

In an embodiment the system further comprises a fitness parameter providing unit and a training impulse variable determination unit. The fatigue index decrement subunit is configured to decrement the fatigue index based on both the training impulse variable and the fitness parameter. In this embodiment, the advantages of both above described embodiments are combined. In the consequence, preferentially the fatigue index is determined more accurately. Preferentially, both the fitness parameter providing unit and the training impulse variable determination unit can be combined with any of the above described embodiments.

In an embodiment the fatigue index decrement subunit is configured to decrease the fatigue index based on an exponential decay function.

In this embodiment, an exponential decay function is referred to as an exponential function having a negative exponent. In other words, the exponential decay function results in a value between 0 and 1. Preferentially, the fatigue index of a directly preceding exercise time is multiplied with the exponential decay factor in order to decrement the fatigue index for the current exercise time. Preferentially, the exponential decay function comprises a function depending on at least one of a fitness parameter representative of a fitness of the subject, a training impulse variable, the heart rate or the heart rate reserve percentage as the exponent.

In an embodiment the system further comprises a weighting function determination unit for determining a weighting function based on the heart rate. The weighting function is configured to determine a weight which increases with the heart rate. The fatigue index determination unit comprises an aerobic fatigue index determination subunit and an anaerobic fatigue index determination subunit. The aerobic fatigue index determination subunit is configured to determine an aerobic fatigue index of the subject based on the heart rate. The anaerobic fatigue index determination subunit is configured to determine an anaerobic fatigue index of the subject based on the heart rate and the weighting function. The fatigue index determination unit is configured to determine the fatigue index as a combination of the aerobic fatigue index and the anaerobic fatigue index.

In an embodiment the system further comprises a training impulse variable determination unit for determining a training impulse variable based on at least one of the heart rate and the heart rate reserve percentage. The aerobic fatigue index decrement subunit is configured to determine the aerobic fatigue index decrement exponentially proportional to the inverse of the training impulse variable. The anaerobic fatigue index decrement subunit is configured to determine the anaerobic fatigue index decrement exponentially proportional to the training impulse variable.

The training impulse variable determination unit can preferentially be provided as any of the above described advantageous embodiments.

The fatigue index determination unit, the delay time determination unit and the fatigue index increment subunit can in one embodiment be provided in one or more processors that are arranged in the same or different physical devices. More precisely, the fatigue index determination unit, the delay time determination unit and the fatigue index increment subunit can in one embodiment be provided together with the exercise timer providing unit and/or the physiological measure providing unit in a single device or in a different embodiment be distributed over multiple devices.

In an embodiment the delay time determination unit, the fatigue index determination unit and/or the fatigue index increment subunit are adapted for communicating with the exercise timer providing unit and/or the physiological measure providing unit in a wired or wireless manner as well as known in the art.

In an embodiment, one, more or all of the exercise timer providing unit, the physiological measure providing unit, the fatigue index determination unit, the delay time determination unit and the fatigue index increment subunit are provided at a server, which is arranged for communicating with the rest of the system for quantifying physical fatigue by suitable communication means, for instance via the Internet.

In an embodiment the system for quantifying physical fatigue allows for an unobtrusive quantification of physical fatigue based on the physiological measure of the subject.

In an embodiment, one or more of the units of the system are provided with a wrist-worn device, such as a watch. In this embodiment the heart rate providing unit preferentially comprises a heart rate sensor, such as an optical heart rate sensor based on photoplethysmography. However, it should be understood that also other embodiments of the system, such as an implementation as a web application, are contemplated.

In a further aspect a method for quantifying physical fatigue of a subject is provided. The method comprises providing a physiological measure of the subject and determining a fatigue index of the subject. The fatigue index comprises a first fatigue index based on the physiological measure and a second fatigue index based on the physiological measure. The first fatigue index and the second fatigue index are determined with a respectively different characteristic based on the physiological measure.

In a further aspect a method for quantifying physical fatigue of a subject is provided. The method comprises: providing an exercise time of the subject, providing a heart rate of the subject corresponding to the provided exercise time, determining a fatigue index of the subject, determining a delay time based on the heart rate, and increasing the fatigue index based on the heart rate, wherein the fatigue index is increased in case the exercise time exceeds the delay time only.

In a further aspect a computer program for quantifying physical fatigue of a subject is provided. The computer program comprises program code means for causing a system as defined in claim 1 to carry out the method as defined in claim 14, when the computer program is run on the system.

It shall be understood that the system of claim 1, the method of claim 14 and the computer program of claim 15 have similar and/or identical preferred embodiments, in particular, as defined in the dependent claims.

It shall further be understood that all aspects of the present invention provide an improvement over the prior art both individually and in combination. It shall further be understood that a preferred embodiment of the present invention can also be any combination of the dependent claims or above embodiments with the respective independent claim of one or several aspects.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system for quantifying physical fatigue of a subject,

FIG. 2 shows schematically and exemplarily a further embodiment of a system for quantifying physical fatigue of a subject,

FIG. 3 shows schematically and exemplarily a flowchart implemented in the system according to FIG. 2,

FIGS. 4A and 4B show schematically and exemplarily the behavior of the system during a first exercise,

FIGS. 5A and 5B show schematically and exemplarily the behavior of the system during a second exercise, and

FIG. 6 shows a flowchart exemplarily illustrating an embodiment of a method for quantifying physical fatigue of a subject for the system for quantifying physical fatigue of a subject shown in FIG. 1 or FIG. 2.

### DETAILED DESCRIPTION OF EMBODIMENTS

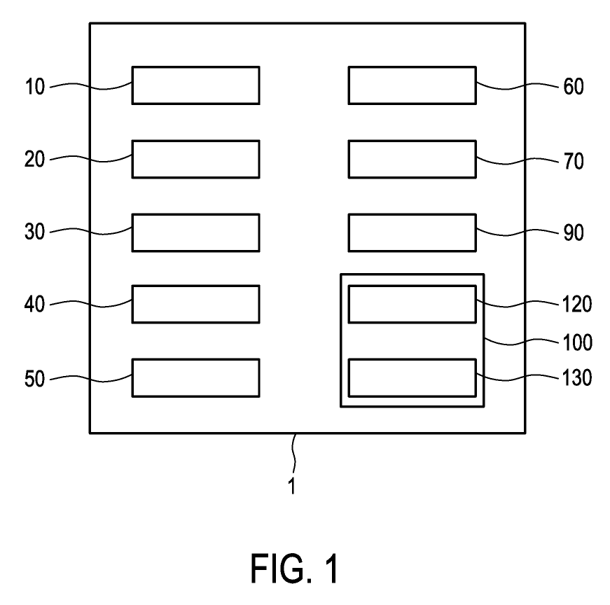

FIG. 1 shows schematically and exemplarily an embodiment of a system 1 for quantifying physical fatigue of a subject. The system 1 comprises an exercise timer providing unit 10, a heart rate providing unit 20, a resting heart rate providing unit 30, a maximum heart rate providing unit 40, a heart rate reserve (HRR) determination unit 50, a delay time determination unit 60, a training impulse variable determination unit 70, a fitness parameter providing unit 90 and a fatigue index determination unit 100. The fatigue index determination unit 100 comprises a fatigue index increment subunit 120 and a fatigue index decrement subunit 130.

Exercise timer providing unit 10 provides a timer signal, which is indicative of a time during which a subject is exercising. In this example, the timer signal is reset every time the subject commences a new exercise or activity and provides a signal indicative of the elapsed time since the beginning of the exercise and/or activity. Exercise timer providing unit 10 can provide the exercise timer signal substantially in real time, i.e. during the exercising of the subject, or based on a previously recorded and stored data set comprising timer data of multiple and consecutive points in time during an exercise of the subject.

Heart rate providing unit 20 provides a heart rate corresponding to the point in time to which exercise timer providing unit 10 provides the exercise time. As with exercise timer providing unit 10, heart rate providing unit 20 can thus substantially provide a heart rate in real time, wherein heart rate providing unit 20 can comprise any suitable means for determining and/or measuring a heart rate of the subject. In another example, heart rate providing unit 20 provides a heart rate of a previously recorded and stored heart rate that is stored in association with the corresponding exercise time. Heart rate providing unit 20 is an example of a physiological measure providing unit, wherein in other examples also other physiological measures can likewise be provided. Expressed differently, while the following example is described using the heart rate as a physiological measure, also a respiration rate, a blood pressure, a body temperature, a blood marker or other physiological measure or other physiological measure can likewise be used in other examples.

Resting heart rate providing unit 30, maximum heart rate providing unit 40 and fitness parameter providing unit 90 provide a resting heart rate, a maximum heart rate and a fitness parameter of the subject, respectively. All of the resting heart rate, the maximum heart rate and the fitness parameter can be stored on a suitable storage unit, such as a local or remote database, received as an input from the subject or be determined based on suitable measurements. For example, the resting heart rate can be determined based on a heart rate provided by heart rate providing unit 20 while the subject is at rest. As a further example, the maximum heart rate can be determined from the highest heart rate determined by heart rate providing unit 20 during the course of one or a plurality of exercises. The fitness parameter preferentially comprises a maximum oxygen uptake (VO2ₘₐₓ), which can be input by the user or determined with a measuring apparatus as known in the art. The fitness parameter can in a different example also be a fitness level ranking based on the subjects answers to a questionnaire. However, also further examples of fitness parameters are of course contemplated.

In summary, the resting heart rate, the maximum heart rate and the fitness parameter can all be provided as an input from the subject, stored with a profile of the subject, for instance with the system 1 itself or at a server, be determined by resting heart rate providing unit 30, maximum heart rate providing unit 40 and fitness parameter providing unit 90 itself, or any combination of the three alternatives.

HRR determination unit 50 determines the heart rate reserve based on the resting heart rate and the maximum heart rate, for instance as a difference between maximum heart rate and resting heart rate. The heart rate reserve is a reliable indicator of the relative intensity of an activity for a particular subject. It is therefore suitable for determining the physical fatigue, which is dependent on the intensity of the exercise.

Delay time determination unit 60 determines a delay time, after which accumulation of fatigue starts. The underlying finding is that fatigue accumulation does not start with the starting of an exercise, but following a delay depending on the intensity of the activity, among others. For example, the time required to start fatiguing is different if the subject is walking or running Fatigue index increment subunit 120 is configured to only increase the fatigue index, i.e. to accumulate fatigue, when the subject is exercising for a time longer than the delay determined by delay time determination unit 60.

In an example, the delay can be calculated as:

$$Delay = A \times Fitness \times \frac{{HR}_{max} - HR ( t )}{HR ( t ) - {HR}_{rest}}$$

In this example, A is a constant, which can be determined, for instance, from empirical measurements. Fitness preferentially can be the fitness parameter provided by fitness parameter providing unit 90. HRₘₐₓ can be the maximum heart rate provided by maximum heart rate providing unit 40 and HRᵣₑₛₜ can be the resting heart rate provided by resting heart rate providing unit 30. HR(t) corresponds to the heart rate provided by heart rate providing unit 20 for a particular exercise time t provided by exercise timer providing unit 10.

The above formula is of course only one example to determine the delay. In this example, the delay is determined to be zero in case the current heart rate is equal to the maximum heart rate, i.e. no delay would be assumed in case of a very intense activity. To the contrary, in case the current heart rate HR(t) approaches the resting heart rate, the delay time increases up to infinity, i.e. no fatiguing is assumed in case the heart rate does not differ from the resting heart rate, i.e. the subject is at rest.

The fatigue index is determined by fatigue index determination unit 100, wherein two independent operations on the fatigue index are performed by fatigue index increment subunit 120, namely the fatigue index being increased, and by fatigue index decrement subunit 130, namely the fatigue index being decreased.

This general principle of increasing and decreasing fatigue leads to five possible states, in which a subject can be. First, the subject can be in an inactive, unfatigued state, such as a sedentary period, which is not preceded by any fatiguing activity. An example of such activity can be working at a desk. A second state can be an unfatigued state, in which the subject is carrying out active unfatiguing, such as walking or cycling at low speed. In the first and second state, the fatigue index of the subject is zero and no increase of the fatigue occurs.

A third state is an active fatiguing state, wherein the subject can be unfatigued or not, for instance the subject can carry out an intense activity such as running, jogging and aerobic training sessions. In this state, the fatigue will increase with an increase rate dependent on the activity intensity and the fitness of the subject.

In the fourth state, the subject can be in an inactive fatigued state, i.e. a sedentary period following a fatiguing exercise, for instance the subject can be sitting after running or having a half time brake during a match. In this state, the fatigue will decrease, while the decrease rate is dependent on the fitness of the subject.

Finally, in a fifth state, the subject can carry out active unfatiguing in a fatigued state, i.e. the subject is performing an unfatiguing activity after a fatiguing exercise, such as intermitted training sessions. In this state, the fatigue will decrease with a decrease rate dependent on the activity intensity and the subject fitness.

Dependent on the state of the subject, fatigue index increment subunit 120 increases the fatigue index and/or fatigue index decrement subunit 130 decreases the fatigue index accordingly.

In this example, fatigue index increment subunit 120 increases the fatigue index based on a training impulse variable determined by training impulse variable determination unit 70. As indicated before, fatigue index increment subunit 120 is configured to only increase the fatigue index in case the exercise time exceeds the determined delay time. Training impulse variable determination unit 70 is configured to determine a training impulse variable (TRIMP), which is indicative of a training intensity for the subject carrying out an exercise at the heart rate provided by heart rate providing unit 20. In one example, training impulse variable determination unit 70 can determine the training impulse variable using the formula:

TRIMP(t)=HRᵣₑₛ(t)×k×e^{b×HR}^{ᵣₑₛ}^{(t)}

HRᵣₑₛ (t) is preferentially the heart rate reserve determined by HRR determination unit 50, k and b are two constants which depend on the gender of the subject. In other examples the mathematical formula can of course be different.

Fatigue index increment subunit 120 can receive the training impulse variable calculated at each time instant and increment the previous fatigue index directly with the determined training impulse variable, e.g. the TRIMP value for a particular time t. In other examples, the training impulse variable can be multiplied with a constant dependent on the sampling frequency prior to being added to the previous fatigue index. Mathematically, this relation can be expressed as:

FI(t)≅FI(t−1)+TRIMP(t)

Fatigue index decrement subunit 130 decrements the fatigue index in this example according to the following formula:

FI(t)=FI(t−1)×Decr[%]

In one example, the decrement term (Decr[%]) is determined for all time periods during the exercise, but is overlaid by the increment term from fatigue index increment subunit 120 in case the subject is still in an active fatiguing state. In other examples, fatigue index decrement subunit 130 can be configured to only determine the decrement contribution in case the time of exercise is lower than the determined delayed time and/or the exercise ends. In all instances, the decrement term determined by fatigue index decrement subunit 130 becomes only prevalent in case the accumulation term becomes or approaches zero. The decrement term is dependent on the training impulse variable in this example, in order to account for both passive and active recovery, and preferentially also on the fitness of the subject. In one example, the decrement term functionally corresponds to an exponential decay function.

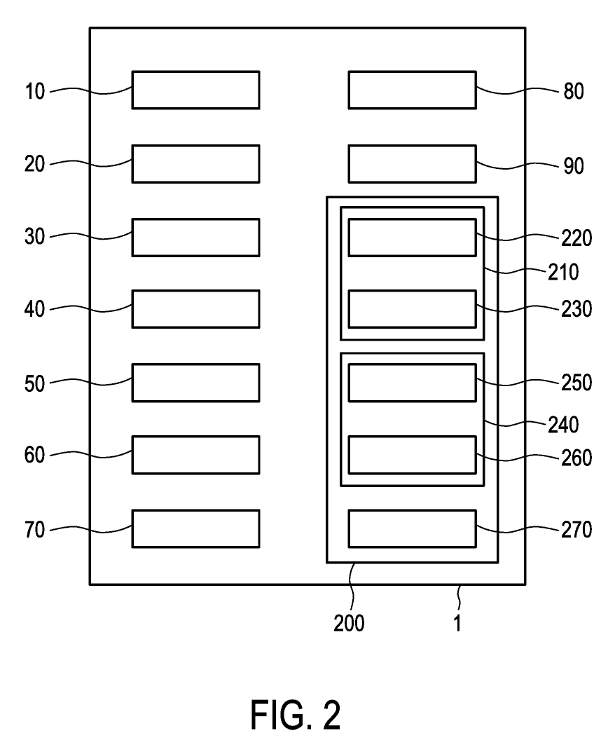

FIG. 2 shows schematically and exemplarily a further embodiment of a system 1 for quantifying physical fatigue of a subject. System 1 as shown in FIG. 2 substantially corresponds to system 1 as shown in FIG. 1, wherein additionally a weighting function determination unit 80 is provided. Instead of fatigue index determination unit 100 a fatigue index determination unit 200 is provided.

Fatigue index determination unit 200 comprises an aerobic fatigue index determination subunit 210, an anaerobic fatigue index determination subunit 240 and a fatigue index combination subunit 270. Each of the aerobic fatigue index determination subunit 210 and the anaerobic fatigue index determination subunit 240 respectively comprises an increment subunit 220, 250 and a decrement subunit 230, 260. In this example, two fatigue indexes, namely an aerobic fatigue index and an anaerobic fatigue index, are determined to more accurately describe the fatigue index of the subject. The aerobic fatigue index determined by aerobic fatigue index determination subunit 210 describes fatigue accumulation mainly during the aerobic phase and the anaerobic fatigue index determined by anaerobic fatigue index determination subunit 240 to describe fatigue accumulation mainly above the aerobic fatigue index, in the anaerobic phase.

The finding underlying this differentiation between aerobic fatigue index and anaerobic fatigue index is that the type of exercise and the relative intensity of such exercise is a fundamental aspect for determining the resulting fatigue, wherein fatigue from high intensity, for instance, results in predominant lactate accumulation given the exertion above anaerobic threshold values, and fatigue from lower intensity exercise results in different physiological phenomena including dehydration and hyperthermia.

Since different origins of fatigue are identified for high intensity and for low intensity exercise, respectively, a different recovery profile for the corresponding fatigue index is applicable. More precisely, in order to recover from a high lactate concentration, exercise at a low intensity can be favorable, while recovery time from a low intensity exercise, i.e. a high aerobic fatigue index, will be hindered by ongoing exercise, even at low intensity. The aerobic and anaerobic fatigue indices are just two examples of a first and a second fatigue index and other fatigue indices, depending on a characteristic of a physiological measure, can be employed in other examples.

Therefore, aerobic fatigue index decrement subunit 230 preferentially accounts for the particular recovery profile of the aerobic fatigue index in that a high heart rate and/or a high training impulse variable leads to a lower decrease contribution. As an example, walking after a running exercise will increase the recovery time of the aerobic fatigue index. To the contrary, anaerobic fatigue index decrement subunit 260 will determine the decrease contribution to the anaerobic fatigue index such that a higher heart rate and/or training impulse variable, will, up to a certain point, lead to a higher decrease. Just to give an example, walking after running will decrease the recovery time of the anaerobic fatigue index, since soft exercising can for instance help in dissolving the lactate from the muscles.

Just to give an example, the decrease contribution of the aerobic fatigue index (LE) and the anaerobic fatigue index (HE) can be determined with the following formulae, wherein of course also different formulae are contemplated:

$${Decrease}_{LE} = {e}^{- \frac{Fitness}{{τ}_{LE} \times TRIMP }}$$

$${Decrease}_{HE} = {e}^{- \frac{Fitness \times TRIMP}{{τ}_{LE}}}$$

τ_{LE} and τ_{HE} preferentially represent the time difference between a previous time and the current time, i.e. the difference between two consecutively provided heart rates or a sampling frequency of the heart rate.

Aerobic fatigue index increment subunit 220 substantially operates the same way as fatigue index increment subunit 120 discussed with reference to FIG. 1. Anaerobic fatigue index increment subunit 250 can rely on the same training impulse variable as aerobic fatigue index increment subunit 220 and weight the training impulse variable with a weighting function provided by weighting function determination unit 80, wherein the weighting function only provides a significant weight in case the exercise intensity is high and thus belongs to an anaerobic range. In other words, the weighting function determined by weighting function determination unit 80 contributes insignificantly until a flex point or threshold is reached, above which a significant contribution and accumulation of anaerobic fatigue is expected. In one example, weighting function determination unit 80 can determine a weighting function based on a sigmoid function, wherein the flex point of the sigmoid can be defined to be in the range of, for instance, 80% of the maximum of the training impulse variable achievable by the subject. However, in other examples, also different functions are contemplated, which fulfill the requirement of putting more weight onto higher exercise intensity, i.e. anaerobic, exercise, such as a step function and the like.

In one example, the function to determine the effect of the aerobic fatigue index (FILE) and the anaerobic fatigue index (FIHE) can mathematically be expressed using the following formulae:

FILE(t)≅FILE(t−1)+TRIMP(t)

FIHE(t)≅FIHE(t−1)+TRIMP(t)×Sigm(TRIMP(t))

Of course, in other examples, also different mathematical relations can be implemented by the person skilled in the art.

Fatigue index combination subunit 270 finally combines the aerobic fatigue index and the anaerobic fatigue index to a global fatigue index. In one basic example, fatigue index combination subunit 270 can add the aerobic fatigue index and the anaerobic fatigue index together, wherein other forms of combinations are contemplated in different examples. Alternatively or additionally, system 1 can also directly and independently provide the aerobic fatigue index determined by aerobic fatigue index determination subunit 210 and the anaerobic fatigue index determined by anaerobic fatigue index determination unit 240. Both the global fatigue index and the aerobic and anaerobic fatigue indices individually can help managing training programs for elite and recreational athletes by helping to avoid overtraining, to dose training intensity, and to estimate recovery time.

It should be noted that not all units described with reference to FIG. 2 are mandatory for the system according to the invention. For instance, an advantageous system can employ the plurality of fatigue indices without the need for delay time determination unit 60, while further advantages however base in the combination.

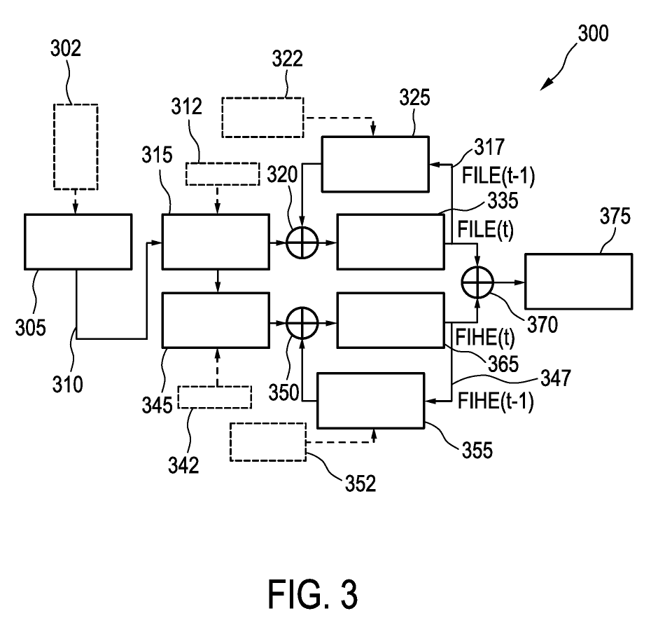

FIG. 3 schematically and exemplarily illustrates a flow chart of an implementation of a three state system 300, which illustrates an exemplary operation of the system 1 described with reference to FIG. 2. The three states of the system 1 are a start state, an increase state and a decrease state, which can be derived from the five possible states a subject can be in as described above. Flow chart 300 is exemplarily described to determine a global fatigue index at a current point in time with reference to a preceding point in time. The preceding point in time is preferentially, without being limited, the point in time directly before the current point in time, e.g. one time step earlier. As also described above, the entire exercise for which the global fatigue index is to be determined can be prerecorded and analyzed at a later stage or can be processed substantially in real time during exercise.

At step 305, a current delay time is determined based on an input 302, wherein the input comprises the heart rate corresponding to the current exercise time, the current exercise time, a fitness parameter such as a maximal oxygen uptake, a resting heart rate, a maximum heart rate, for instance. Alternative and/or additional parameters can of course also be provided as input 302 to the determination the delay function in step 305. Depending on the determined delay time 305, in connection 310 it is decided whether the current exercise time is larger than the delay time and only in case the current exercise time is longer than the delay time, a switch is activated and an increase of the fatigue index is determined in step 315.

The fatigue index increase in step 315 is determined based on an input 312, which comprises, for instance, a HRR percentage for the current point of time. As described in detail above, the fatigue index increase in step 315 can therefore be determined based on a training impulse variable comprised in input 312, for instance.

At step 320, the increment determined in step 315 is summand with a previous aerobic fatigue index 317, i.e. the aerobic fatigue index at the directly preceding point in time, which has been decreased in step 325.

In step 325, the preceding aerobic fatigue index 317 is accordingly decreased based on an input 322 comprising, for instance, the HRR percentage at the current time and the fitness parameter.

Accordingly, at step 320, the directly preceding aerobic fatigue index, which has been decreased, is summand with the increment determined in step 315 to obtain the aerobic fatigue index at the current time in step 335.

Additionally, the fatigue index increased determined in step 315 is subject to a weighting correction in step 345, wherein the weighting correction accounts for the intensity of the activity and weights a determined fatigue index increment with a higher weight in case the exercise is carried out in an anaerobic region. In other words, in step 345, the increase of the anaerobic fatigue index is determined. To this end, an input 342 comprising, for instance, the heart rate reserve percentage at the present time is processed.

The anaerobic fatigue index increment determined in step 345 is summand in step 350 with the previous anaerobic fatigue index 347, which has been decremented in step 355. The decrement in step 355 is implemented in the same way as in step 325 for the aerobic fatigue index and employs an input 352 comprising also the heart rate reserve percentage at the present time and the fitness parameter, for instance. After the summation in step 350, the anaerobic fatigue index is determined in step 365. Finally, the aerobic fatigue index and the anaerobic fatigue index, respectively for the current point in time, can be summand in step 370 to obtain a global fatigue index at the present point in time at step 375.

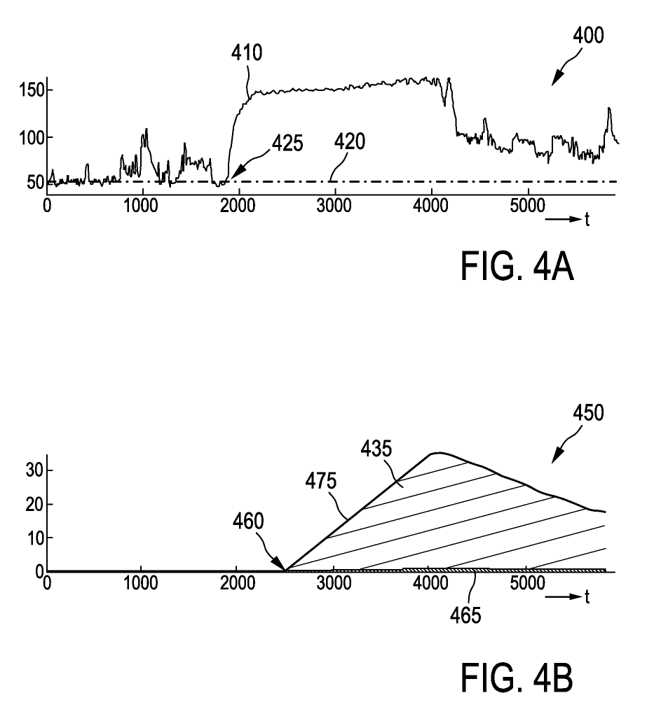

FIG. 4A and FIG. 4B schematically and exemplarily illustrate one exercise session of the subject. On the respective horizontal axis a time of the exercise is indicated, for instance, in seconds.

FIG. 4A illustrates a graph 400, in which a heart rate 410 of the subject during exercise is illustrated on the vertical axis over time on the horizontal axis. As a reference, a resting heart rate 420 is provided. It can be seen that the heart rate of the subject was rather low up to a point in time 425, after which the heart rate maintained a higher level for a significantly long duration.

In FIG. 4B the development of the aerobic fatigue index 435, the anaerobic fatigue index 465 and the global fatigue index 475 is drawn over the same time interval illustrated in FIG. 4A. It can be seen that due to the effect of the delay time, a fatigue onset occurs at position 460, which is later than point 425, at which the subject started exercising at increased intensity. In can further be seen that, since the subject's exercising is at rather low intensity, the global fatigue index 475 substantially corresponds to the aerobic fatigue index 435, while no significant anaerobic fatigue 465 develops.

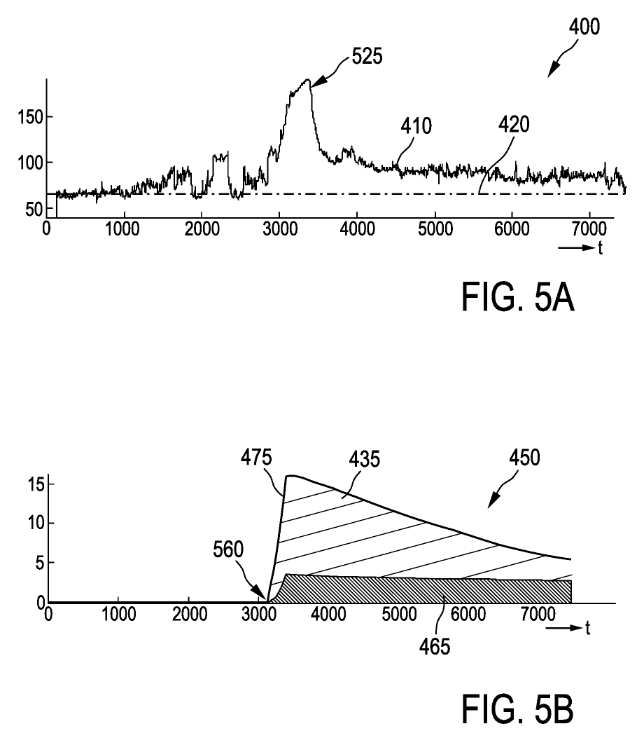

FIGS. 5A and 5B illustrate a further exercise of the subject the same way as illustrated in FIGS. 4A and 4B, wherein the subject carried out a high intensity exercise in the example of FIGS. 5A and 5B. Two effects of the present invention can be seen when comparing FIGS. 5A and 5B with FIGS. 4A and 4B. At time point 525 in FIG. 5A the subject started performing a high intensity exercise. The time difference between onset of the high intensity activity and the accumulation of fatigue, i.e. the delay time, is shortened compared to FIGS. 4A and 4B. In other words, a higher intensity leads to a shorter delay time. In FIG. 5B, fatigue accumulation therefore starts already at a time point indicated with 560. Next, due to the high intensity exercise, a significant contribution of the anaerobic fatigue index 465 can be detected. The anaerobic fatigue index 465 and the aerobic fatigue index 435 can be individually analyzed and corresponding recovery profiles implemented, as described above.

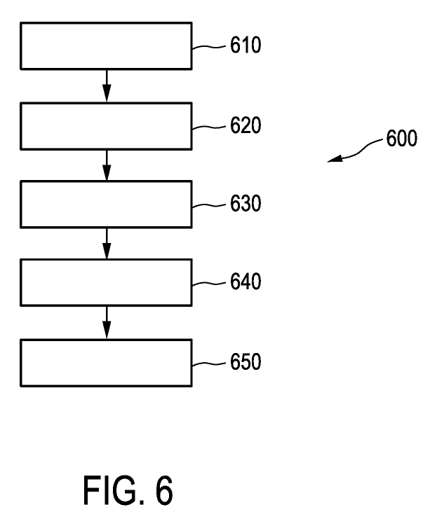

FIG. 6 shows a flow chart exemplarily illustrating an embodiment of a method 600 for quantifying physical fatigue of a subject for the system 1 for quantifying physical fatigue of a subject shown exemplarily in FIGS. 1 and 2.

In step 610, an exercise time of the subject is provided, i.e. a time after the starting of the exercise is provided. In one example, the exercise time is provided by exercise timer providing unit 10.

In step 620, a heart rate of the subject corresponding to the exercise time provided in step 610 is provided as an example of a physiological measure, for instance by means of heart rate providing unit 20.

In step 630, a fatigue index of the subject is determined. In one example, the fatigue index can be a previously determined fatigue index, for instance a fatigue index corresponding to the directly preceding point of time. The determined fatigue index, can, for instance, optionally be decreased based on the principles described above. In one example, an aerobic and an anaerobic fatigue index are determined in step 630 as examples for a first and second fatigue index, wherein the two indices have a respectively different characteristic based on the physiological measure.

In step 640, a delay time is determined based on the heart rate provided in step 620. In one example, the delay time is provided by delay time determination unit 60.

In step 650, the fatigue index is increased based on the heart rate, for instance by means of fatigue index increment subunit 120, aerobic fatigue index increment subunit 220 and/or anaerobic fatigue index increment subunit 250. The fatigue index is increased in case and only in case the exercise time provided in step 610 exceeds the delay time determined in step 640. In other words, it is first compared whether the exercise time exceeds the delay time and then the fatigue index determined in step 630 is increased in step 650 based on the result of this comparison. Thereby, long, low intensity activities can also result in a reliable fatigue index.

It should be noted that the above examples are described with respect to the heart rate as a physiological measure. However, in other examples also other physiological measure providing units can be employed instead of heart rate providing unit 20. Further, although aerobic fatigue index determination subunit and anaerobic fatigue index determination subunit are described as examples for a first and second fatigue index determination subunit, also different first and second fatigue indices, for instance depending on other characteristics of the physiological measure, are contemplated.

Exercise timer providing unit 10, heart providing unit 20, resting heart rate providing unit 30, maximum heart rate providing unit 40, heart rate reserve determination unit 50, delay time determination unit 60, training impulse variable determination unit 70, weighting function determination unit 80, fitness parameter providing unit 90, fatigue index determination unit 100 including fatigue index increment subunit 120 and fatigue index decrement subunit 130, and fatigue index determination unit 200 including aerobic fatigue index determination subunit 210, aerobic fatigue index increment subunit 220, aerobic fatigue index decrement subunit 230, anaerobic fatigue index determination subunit 240, anaerobic fatigue index increment subunit 250, anaerobic fatigue index decrement subunit 260, and fatigue index combination subunit 270 can, in one example, be implemented on a sports watch and/or a sport tracking application, which can be installed on a mobile phone, for instance. However, in other examples, one, more or all of the previously mentioned units and/or subunits can be implemented on a server and accessed, for instance, via a web interface using a mobile phone, a portable and/or stationary computer device. In this example, data provided by exercise timer providing unit 10, heart rate providing unit 20, resting heart rate providing unit 30, maximum heart rate providing unit 40 and fitness parameter providing unit 90 can be stored on a database on the server.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

1. A system for quantifying physical fatigue of a subject,
wherein the system comprises:
a physiological measure providing unit for providing a physiological measure of the subject; and
a fatigue index determination unit Get for determining a fatigue index of the subject,
wherein the fatigue index determination unit comprises a first fatigue index determination subunit for determining a first fatigue index based on the physiological measure and a second fatigue index determination subunit for determining a second fatigue index based on the physiological measure, and
wherein the first fatigue index and the second fatigue index have a respectively different characteristic based on the physiological measure.

2. The system according to claim 1, wherein the physiological measure is indicative of an intensity of a physical activity of the subject.

3. The system according to claim 1, wherein the first fatigue index is an aerobic fatigue index indicative of an aerobic fatigue of the subject and the second fatigue index is an anaerobic fatigue index indicative of an anaerobic fatigue of the subject.

4. The system according to claim 1, wherein the first fatigue index determination subunit comprises a first fatigue index increment subunit for increasing the first fatigue index and a first fatigue index decrement subunit decreasing the first fatigue index, and
wherein the second fatigue index determination subunit comprises a second fatigue index increment subunit for increasing the second fatigue index and a second fatigue index decrement subunit for decreasing the second fatigue index, and
wherein the first fatigue index decrement subunit and the second fatigue index decrement subunit are configured to determine the first fatigue index decrement and the second fatigue index decrement with a respectively different dependency on the physiological measure.

5. The system according to claim 4, further comprising a weighting function determination unit for determining a weighting function based on the physiological measure, wherein the weighting function is configured to determine a weight which increases with an activity intensity indicated by the physiological measure,
wherein the second fatigue index determination subunit configured to determine the second fatigue index based on the physiological measure and the weighting function.

6. The system according to claim 4, further comprising an exercise timer providing unit for providing an exercise time of the subject,
wherein the physiological measure providing unit is configured to provide the physiological measure corresponding to the provided exercise time.

7. The system according to claim 6, further comprising a delay time determination unit for determining a delay time based on the physiological measure,
wherein at least one of the first end second fatigue index increment subunits is configured to increase the fatigue index in case the exercise time exceeds the delay time only.

8. The system according to claim 7, further comprising a fitness parameter providing unit for providing a fitness parameter representative of a fitness of the subject,
wherein the delay time determination unit is configured to determine the delay time based on the fitness parameter and additionally or alternatively at least one of the first fatigue index decrement subunit and the second fatigue index decrement subunit is configured to decrease the fatigue index based on the fitness parameter.

9. The system according to claim 4, wherein the physiological measure providing unit comprises a heart rate providing unit for providing a heart rate of the subject, the system further comprising:
a resting heart rate providing unit for providing a resting heart rate of the subject;
a maximum heart rate providing unit for providing a maximum heart rate of the subject; and
a heart rate reserve determination unit for determining a heart rate reserve based on the resting heart rate and the maximum heart rate,
wherein the heart rate providing unit is further configured to provide the heart rate of the subject in proportion to the heart rate reserve as a heart rate reserve percentage.

10. The system according to claim 9, further comprising a training impulse variable determination unit for determining a training impulse variable based on the physiological measure, in particular based on at least one of the heart rate and the heart rate reserve percentage,
wherein at least one of the first fatigue index increment subunit, the first fatigue index decrement subunit, the second fatigue index increment subunit and the second fatigue index decrement subunit is configured to respectively increment or decrement the fatigue index based on the training impulse variable.

11. The system according to claim 10, wherein the first fatigue index decrement subunit is configured to determine the first fatigue index decrement exponentially proportional to the inverse of the training impulse variable, and the second fatigue index decrement subunit is configured to determine the second fatigue index decrement exponentially proportional to the training impulse variable.

12. The system according to claim 10, wherein the training impulse variable determination unit is configured to determine the training impulse variable based on a product of the heart rate reserve percentage and an exponential of the heart rate reserve percentage and preferably further based on at least one constant depending on a gender of the subject.

13. A method for quantifying physical fatigue of a subject,
wherein the method comprises:
providing a physiological measure of the subject; and
determining a fatigue index of the subject
wherein the fatigue index comprises a first fatigue index based on the physiological measure and a second fatigue index based on the physiological measure, and
wherein the first fatigue index and the second fatigue index are determined with a respectively different characteristic based on the physiological measure.

14. A computer program for quantifying physical fatigue of a subject, the computer program comprising program code means for causing a system as defined in claim 1 to perform the following when the computer program is run on the system:
providing a physiological measure of the subject; and
determining a fatigue index of the subject
wherein the fatigue index comprises a first fatigue index based on the physiological measure and a second fatigue index based on the physiological measure, and
wherein the first fatigue index and the second fatigue index are determined with a respectively difference characteristic base don the physiological measure.
